(19) United States
(12) Patent Application Publication
(10) Pub. No.: US 2023/0249127 A1
(43) Pub. Date: Aug. 10, 2023

(54) RATE ENHANCEMENT OF GAS CAPTURE PROCESSES

(71) Applicant: C-Capture Ltd, Leeds (GB)

(72) Inventors: Douglas Barnes, Leeds (GB); Caspar Schoolderman, Leeds (GB); Duncan Holdsworth, Leeds (GB); Christopher Rayner, Leeds (GB)

(73) Assignee: C-Capture Ltd, Leeds (GB)

(21) Appl. No.: 18/015,898

(22) PCT Filed: Jul. 14, 2021
(86) PCT No.: PCT/GB2021/051805

§ 371 Date: Jan. 12, 2023

(30) Foreign Application Priority Data
Jul. 15, 2020 (GB) 2010908.8

(51) Int. Cl. B01D 53/62; B01D 53/14; B01D 53/78; B01D 53/96; B01D 53/84; C12N 9/88

(52) U.S. Cl. CPC B01D 53/62; B01D 53/78; B01D 53/84; B01D 53/96; B01D 53/1425; B01D 53/1475; B01D 53/1493; C12N 9/88; C12Y 402/01001; B01D 2251/80; B01D 2251/306; B01D 2252/103; B01D 2252/504; B01D 2252/2025; B01D 2255/804; B01D 2257/504; B01D 2258/0283

(57) ABSTRACT

The present invention relates to a method of capturing CO₂ from a gas stream. The method uses a two liquid phase capture composition.

### FIELD OF THE INVENTION

The present invention relates to a method of capturing CO₂ from a gas stream. The method uses a two phase liquid capture composition.

### BACKGROUND TO THE INVENTION

Climate change as a result of human activity is a threat to the health and prosperity of billions of people around the world. With the 2015 Paris Agreement the international community agreed to keep the global temperature rise this century well below 2° C. above pre-industrial levels and to pursue efforts to limit the temperature increase even further to 1.5° C. A significant contributor to climate change is CO₂, released in enormous quantities by many industries and particularly by the generation of energy by combustion, either of fossil fuels or of biofuels. In order to meet the Paris targets, we have to reimagine our whole approach to energy provision on a global scale.

Carbon capture technology allows energy systems and industrial processes to be decarbonised in the short term, buying time to replace them with carbon neutral alternatives, and variations such as BECCS (Biomass Energy with Carbon Capture & Storage) have the potential to facilitate negative CO₂ emissions so as to offset the emissions from sectors which cannot be easily decarbonised with current technology, for example, aviation.

WO2015/092427 describes methods of carbon capture using compositions that comprise a mix of miscible solvents.

### SUMMARY OF THE INVENTION

In a first aspect of the present invention is a method for capturing CO₂ from a gas stream, the gas stream containing CO₂, the method comprising:
contacting the gas stream containing CO₂ with a capture composition in a gas-liquid contacting apparatus to generate a loaded capture composition, the capture composition comprising:
   i.a first liquid phase comprising at least one capture reagent; and
   ii.a second liquid phase;
wherein the second liquid phase is chemically inert to CO₂. The second phase will typically be an effective solvent for CO₂. It may be that the solubility (e.g. physical solubility) of CO₂ in the second liquid phase is greater than the solubility (e.g. physical solubility) of CO₂ in the first liquid phase.

The first and second liquid phases are separate phases, i.e. the capture composition comprises two liquid phases. Thus, the components of the two phases and the relative quantities at which those components are present will be selected such that the capture composition comprises two liquid phases.

The invention stems from an appreciation on the part of the inventors of the physical, as well as chemical, steps of the CO₂ capture mechanism. The first step of the capture mechanism is the physical dissolution of CO₂ into the capture composition comprising the capture reagent. Once the CO₂ has physically dissolved into the capture composition, it is then able to undergo chemical reaction with the capture reagent. The rate at which the CO₂ dissolves into the capture composition will be governed, in part, by the availability of CO₂, i.e. its volumetric concentration (partial pressure), in the gas phase and therefore the availability of CO₂ for dissolution/reaction at the gas-liquid phase boundary.

The inventors have found that the use of two liquid phases enhances the rate of CO₂ capture. Without wishing to be bound by theory, by incorporating a second liquid phase which is a more effective solvent for CO₂ than the first liquid phase, the physical elements of the capture mechanism may be altered slightly but importantly. With the second liquid phase present, the CO₂ can initially dissolve into this, and may do so to a greater concentration than it is found in the gas phase, it may then pass into the first liquid phase whereupon it can react with the capture reagent. The overall effect of the second liquid phase may be to make the CO₂ more available to the capture reagent in the first liquid phase, particularly at the liquid-liquid phase boundary, thus increasing the overall rate of CO₂ absorption. In essence, the second liquid phase may act as a means of concentrating the CO₂ prior to it undergoing dissolution into, and reaction in, the first liquid phase comprising at least one capture reagent. In addition, it may be that the second phase increases the surface area of the first liquid phase being exposed to CO₂, which is of high importance for fast reactions and kinetically enhanced mass transfer, where mass transfer rate becomes directly proportional to interfacial surface area. Furthermore, it may be that where the two liquid phases are partially miscible the interfacial mass transfer resistance might be further reduced by reducing the interfacial resistance.

In a second aspect of the invention is provided a method for reacting a gas from a gas stream with a reactive liquid, the gas stream containing the gas, the method comprising:
contacting the gas stream containing the gas with a composition in a gas-liquid contacting apparatus to generate a product composition, the composition comprising:
   i. a first liquid phase comprising a reagent for reacting with the gas; and
   ii. a second liquid phase;
wherein the second liquid phase is chemically inert to the gas.

The second phase will typically be an effective solvent for CO₂. It may be that the solubility of CO₂ in the second liquid phase is greater than the solubility of CO₂ in the first liquid phase.

The first and second liquid phases are separate phases, i.e. the composition comprises two liquid phases. Thus, the components of the two phases and the relative quantities at which those components are present will be selected such that the composition comprises two liquid phases.

The principals established by the inventors on CO₂ capture systems apply equally to other gas liquid reaction systems.

The first liquid phase and the second liquid phase may be immiscible. The first liquid phase and the second liquid phase may be partially miscible. Where they are partially miscible, they will not be fully miscible under the conditions (i.e. at the temperature, pressure and relative proportions of the two liquid phases) of the gas-liquid contacting step.

Chemical reactions involving both gas phase and liquid (or solution) phase reagents are particularly challenging at process scale due to the difficulty of bringing the various components of the reaction together. Various gas-liquid contactors have been developed to solve this problem including, but not limited to; packed columns (with random or structured packings, in co-flow, counter-flow, or cross-flow configurations), spray towers, plate or tray columns, stirred tank reactors (in both continuous and batch configurations), tubular flow reactors (under both laminar and turbulent flow conditions), bubble column reactors, falling film reactors, and membrane reactors. More recent developments are spinning disc and rotating packed bed reactors.

For a given application, the selection criteria for choosing the optimal reactor will be specific to that application and designing to these criteria forms part of the skill of the process engineer. Examples of critical design criteria might include, but not be limited to; minimising the capital cost of the plant, or maximising the reaction yield efficiency of the plant, or minimising the electrical energy demand of the plant. For those applications where it is appropriate, process intensification, defined as “targeted improvement of a process at the unit operations, tasks and phenomena scales in order to increase process efficiency and improve sustainability” (Anantasarn N., Babi D. K., Suriyaprapphadilok U., Gani R., Comput. Aided Chem. Eng., 2016, 38, 1093-1098, https://doi.org/10.1016/B978-0-444-63428-3.50187-9), is an active area of process engineering research and development.

The gas-liquid contacting step may be carried out at a total gas pressure from 0.1 to 200 Bar(abs). The gas-liquid contacting step may be carried out at a total gas pressure from 0.1 to 100 Bar(abs). The gas-liquid contacting step may be carried out at a total gas pressure from 0.1 to 50 Bar(abs).

The gas-liquid contacting step may be carried out at a temperature from 0 to 200° C. The gas-liquid contacting step may be carried out at a temperature from 0 to 100° C. The gas-liquid contacting step may be carried out at a temperature from 20 to 50° C.

The optimal proportion of the second liquid phase required to effect a performance enhancement will be dependent upon the manner in which the gas-liquid contact process is undertaken. Typically the second liquid phase will be present in an amount in the range from 1 to 80% of the overall volume of the composition (i.e. the capture composition). The second liquid phase may be present in an amount in the range from 10 to 70% of the overall volume of the composition (i.e. the capture composition). The second liquid phase may be present in an amount in the range from 40 to 60% of the overall volume of the composition (i.e. the capture composition). The second liquid phase may be present in an amount in the range from 20 to 50% of the overall volume of the composition (i.e. the capture composition).

Thus, it may be that the ratio of the first liquid phase to the second liquid phase may be in the range of 99:1 to 1:4 by volume in the overall volume of the composition (i.e. the capture composition). It may be that the ratio of the first liquid phase to the second liquid phase may be in the range of 9:1 to 1:3 by volume in the overall volume of the composition (i.e. the capture composition). It may be that the ratio of the first liquid phase to the second liquid phase may be in the range of 3:2 to 2:3 by volume in the overall volume of the composition (i.e. the capture composition). It may be that the ratio of the first liquid phase to the second liquid phase may be in the range of 4:1 to 1:1 by volume in the overall volume of the composition (i.e. the capture composition).

### CO₂ Capture

The preceding discussion applies to all claimed methods of reacting a gas from a gas stream with a reactive liquid, including the CO₂ capture methods of the first aspect of the invention. The following discussion relates specifically to the CO₂ capture methods of the first aspect of the invention.

The first liquid phase may comprise water. The first liquid phase may be an aqueous solution comprising the at least one capture reagent.

The at least one capture reagent may be at least one salt of at least one carboxylic acid. The first liquid phase may be an aqueous solution of at least one salt of at least one carboxylic acid.

The at least one salt of at least one carboxylic acid may be at least one metal salt. The metal cation may be selected from alkali metal, alkali earth metal, i.e. Groups 1 & 2 of the periodic table, and mixtures thereof. It may be that the salt of a carboxylic acid is a salt of an alkali metal such as lithium, sodium or potassium. It may be that the salt of a carboxylic acid is a potassium salt.

Salts of aliphatic, aromatic, or heteroaromatic carboxylic acids (e.g. salts of aliphatic or aromatic carboxylic acids) may be used for the purposes of the invention. Suitable aliphatic carboxylic acids may be selected from straight chained, branched or cyclic carboxylic acids which may be saturated or unsaturated, substituted or unsubstituted by substituent groups, heteroatoms, aromatic or heteroaromatic rings systems. The carboxylic acid or each carboxylic acid may comprise a single carboxylic acid group. Polycarboxylic acids, such as di, tri or tetra carboxylic acids, are also suitable as are polymeric acids, such as polyacrylic and polymethacrylic acids, and naturally derived biopolymeric carboxylic acids, for example alginic acid (from seaweed) and pectin (from plant cell walls). Salts of aromatic or heteroaromatic carboxylic acids, such as benzoic acid, are also suitable for the purposes of the invention. Such salts may be in solution, slurry or dispersion.

Typically, said at least one carboxylic acid is at least one C₁-C₂₀ aliphatic carboxylic acid, more typically at least one C₁-C₈ aliphatic carboxylic acid or at least one C₁-C₆ aliphatic carboxylic acid. Said aliphatic carboxylic acid(s) may be straight chained or branched. Exemplary acids include acetic acid, propionic acid, butyric acid (including both n-butyric acid and iso-butyric acid), pentanoic acid (including both n-pentanoic acid and branched pentanoic acids, e.g. pivalic acid) and hexanoic acid (including both n-hexanoic acid and branched hexanoic acids). In certain specific embodiments, the salt is a potassium salt of an aliphatic carboxylic acid, e.g. the potassium salt of a C₁-C₆ aliphatic carboxylic acid that may be straight chained or branched.

It may be that the first liquid phase comprises a single salt of a carboxylic acid. It may be that first liquid phase comprises a mixture of two or more salts of carboxylic acids. Where the first liquid phase comprises a mixture of two or more salts, it may be that the two or more salts will comprise the same cationic counterion but be derived from different carboxylic acids and/or it may be that the two or more salts are derived from the same carboxylic acid but with varying cationic counterions. It may be a mixture of at least one salt of a C₁-C₄ aliphatic carboxylic acid, that may be straight chained or branched, and at least one salt of a C₅-C₆ aliphatic carboxylic acids, that may be straight chained or branched. It may be that both salts are potassium salts of the indicated carboxylic acids.

It may be that the aliphatic carboxylic acids, that may be straight chained or branched, are not substituted by substituent groups that include heteroatoms, i.e. atoms which are not C or H. It may be that the aliphatic carboxylic acids are not substituted by substituent groups that include nitrogen. Aliphatic carboxylic acids may, however, be substituted by a substituent group that comprises O, e.g. a hydroxyl group, an alkoxy group, or an aryloxy group. An illustrative example is lactic acid. The at least one carboxylic acid may comprise only carbon, hydrogen and oxygen.

It may be that the carboxylic acid is not an amino acid or each carboxylic acid is not an amino acid. It may be that the carboxylic acid does not comprise nitrogen or each carboxylic acid does not comprise nitrogen. It may be that the capture composition is substantially free of amino acids. It may be that the capture composition is substantially free of any organic compounds that comprise nitrogen. The term ‘substantially free’ may be considered to mean that no more than 25% by weight of the capture composition is amino acid or an organic compound that comprises nitrogen. It may mean that no more than 10% by weight of the capture composition is amino acid or an organic compound that comprises nitrogen. It may mean that no more than 5% by weight of the capture composition is amino acid or an organic compound that comprises nitrogen. It may mean that no more than 2% by weight of the capture composition is amino acid or an organic compound that comprises nitrogen. It may mean that no more than 1% by weight of the capture composition is amino acid or an organic compound that comprises nitrogen.

The at least one capture reagent (i.e. the at least one salt of at least one carboxylic acid) may be present in the first liquid phase at a concentration in the range 0.5 M to 15 M. The at least one capture reagent (i.e. the at least one salt of at least one carboxylic acid) may be present in the first liquid phase at a concentration in the range 1 M to 15 M. The at least one capture reagent (i.e. the at least one salt of at least one carboxylic acid) may be present in the first liquid phase at a concentration in the range 2 M to 15 M. The at least one capture reagent (e.g. the at least one salt of at least one carboxylic acid) may be present in the first liquid phase at a concentration in the range 3 M to 12 M. The at least one capture reagent (e.g. the at least one salt of at least one carboxylic acid) may be present in the first liquid phase at a concentration in the range 4 M to 9 M. These concentrations are the concentrations in the first liquid phase not in the capture composition as a whole.

It may be that the first liquid phase comprises an aqueous solution of the at least one capture agent (i.e. the at least one salt of at least one carboxylic acid). It may be that the first liquid phase comprises an aqueous solution of an alkali metal salt of a C₂-C₅-aliphatic carboxylic acid. It may be that the aqueous solution has a concentration such that the molar ratio of salt:water is in the range of 1:2.5 to 1:15. It may be that the aqueous solution has a concentration such that the molar ratio of salt:water is in the range of 1:2.5 to 1:12.5. It may be that the aqueous solution has a concentration such that the molar ratio of salt:water is in the range of 1:2.5 to 1:10. It may be that the aqueous solution has a concentration such that the molar ratio of salt:water is in the range of 1:2.5 to 1:7.5. It may be that the aqueous solution has a concentration such that the molar ratio of salt:water is in the range of 1:2.5 to 1:5.

It may be that the first liquid phase further comprises a base additive.

The base additive may be present in the first liquid phase at a concentration in the range 1 M to 10 M. The base additive may be present in the first liquid phase at a concentration in the range 1.5 M to 6 M. The base additive may be present in the first liquid phase at a concentration in the range 2 M to 4 M. These concentrations are the concentrations in the first liquid phase not in the capture composition as a whole.

The base additive is a chemical species that is capable of deprotonating the carboxylic acid from which the salt is derived under the conditions at which the CO₂ capture reaction is performed. Thus, the base additive is a species having a conjugate acid that has a higher pKa under the conditions at which the CO₂ capture reaction is performed than the carboxylic acid from which the salt is derived. The base additive may have a pKa of between 5 and 14 as measured in a dilute aqueous solution. In these embodiments, it may be that the salt of a carboxylic acid is a salt of a C₅-C₈ aliphatic carboxylic acid that may be straight chained or branched. It may be that the salt of a carboxylic acid is a potassium salt of a C₅-C₈ aliphatic carboxylic acid that may be straight chained or branched.

The base additive may be a salt. It may therefore be that the base additive is not a nitrogen base (i.e. does not comprise an amine).

The base additive may be a carbonate salt. The carbonate salt may be an alkali metal or alkali earth metal, i.e. Groups 1 & 2 of the periodic table, carbonate and mixtures thereof. The carbonate salt may be a potassium, sodium, lithium, magnesium or calcium carbonate, or a combination thereof.

Where the base additive is a salt (e.g. a carbonate or a salt of a phenol), the salt may comprise the same cationic counterion as the at least one salt of the at least one carboxylic acid. The base additive may be a potassium salt.

It may be that the first liquid phase further comprises an enzyme. The enzyme accelerates the capture of the CO₂ and/or release of the CO₂. It may do this by catalysing the hydration of carbon dioxide to form carbonic acid and salts thereof, i.e. carbonates and bicarbonates, and/or catalysing the dehydration of carbonic acid and salts thereof, i.e. carbonates and bicarbonates, to form carbon dioxide. Thus the enzyme may be a carbonic anhydrase. It may be that the enzyme is a natural, or native, carbonic anhydrase. It may be that the enzyme is a carbonic anhydrase that has been engineered to optimise its performance as an accelerant in the capture composition. Examples of engineered carbonic anhydrases include those that have been chemically and biochemically modified relative to the natural carbonic anhydrase. This may mean that the natural enzyme is obtained and itself subjected to modification or it may mean that a modified enzyme is produced from component parts.

The enzyme may be present in the first liquid phase at a concentration in the range from 0.01 to 5 g/L, e.g. 0.5 to 1.5 g/L. These concentrations are the concentrations in the first liquid phase not in the capture composition as a whole.

The first liquid phase may comprise an ionic liquid.

The first liquid phase does not necessarily need to be a single, pure solvent but could be a mix of two or more compounds so long as the mixture as a whole meets the conditions set out above. Depending on the nature or identity of the first liquid phase, other minor components may need to be incorporated to aid management of the capture process, for example an anti-oxidant to prevent or reduce oxidative degradation of the first liquid phase components or perhaps an anti-foaming agent to prevent or reduce foaming of the first liquid phase in the a gas-liquid contactor. Suitable species to fulfil these roles will be well known to those skilled in the art and do not form the basis of this invention. The nature, identity and quantity of these minor components would be specific to the capture application and the identity of the second liquid phase.

It may be that the solubility (e.g. physical solubility) of CO₂ in the second liquid phase is greater than the solubility (e.g. physical solubility) of CO₂ in the first liquid phase.

The second liquid phase may comprise at least one organic solvent. The at least one organic solvent will be chosen such that it is immiscible or partially miscible with the first liquid phase. The at least one organic solvent may comprise only hydrogen, silicon, carbon and oxygen atoms, e.g. only hydrogen, carbon and oxygen atoms. The at least one organic solvent may comprise at least one oxygen atom. Classes of organic solvents that typically have high solubility for CO₂ include: the ethers, including cyclic and acyclic ethers, polyethers, such as ethylene glycol ethers, propylene glycol ethers and butylene glycol ethers and their terminally alkylated derivatives, the silicones/siloxanes, including cyclic and acyclic variants, hydrocarbons, such as alkanes, cycloalkanes, arenes, alkylated arenes and their perfluorinated derivatives, and carbonyl compounds such as esters, lactones, ketones and aldehydes. It may be that the at least one organic solvent is selected from silicones, siloxanes and ethers.

Illustrative solvents that are suitable for use in the methods of the invention when the first liquid phase is an aqueous solution include, but are not limited to: 1,2-dimethoxyethane, 1,2-diethoxyethane, ethylene glycol methyl ethyl ether, diethylene glycol dimethyl ether, diethylene glycol diethyl ether, diethylene glycol methyl ethyl ether, triethylene glycol dimethyl ether, triethylene glycol diethyl ether, triethylene glycol methyl ethyl ether, 1,2-dimethoxypropane, 1,2-diethoxypropane, dipropylene glycol dimethyl ether, dipropylene glycol diethyl ether, dipropylene glycol methyl ethyl ether, tripropylene glycol dimethyl ether, tripropylene glycol diethyl ether, tripropylene glycol methyl ethyl ether, di-isopropyl ether, dibutyl ether, ethyl butyl ether, methyl tert-butyl ether, tetrahydrofuran, 2-methyltetrahydrofuran, octamethyltrisiloxane, decamethyltetrasiloxane, hexamethylcyclotrisiloxane, octamethylcyclotetrasiloxane, butyl acetate, pentyl acetate, pentyl propionate, hexyl propionate, hexyl butyrate, heptyl butyrate, gamma-butyrolacetone, gamma-octanoic lactone, 2-pentanone, 3-heptanone, 4-octanone, hexanal, heptanal, octanal, decanal.

The second liquid phase does not necessarily need to be a single, pure solvent but could be a mix of two or more compounds so long as the mixture as a whole meets the conditions set out above. Depending on the nature or identity of the second liquid phase, other minor components may need to be incorporated to aid management of the capture process, for example to mitigate foaming, solvent degradation or corrosion. The nature, identity and quantity of these minor components would be specific to the capture application and the identity of the first liquid phase.

Typically, the second liquid phase will comprise only components that are chemically inert to CO₂ under the conditions at which the CO₂ capture reaction is performed.

The capture composition may comprise:
i) A first liquid phase, said first liquid phase comprising an aqueous solution of an alkali metal salt of a C₂-C₅-aliphatic carboxylic acid; wherein the solution has a concentration such that the molar ratio of salt:water in the range 1:2.5 to 1:15;
ii) A second liquid phase, said second liquid phase comprising a solvent of formula (I) or a mixture of more than one solvent of formula (I):

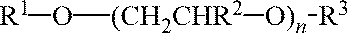

wherein R¹ and R³ are each independently unsubstituted C₁-C₄-alkyl;
R² is independently at each occurrence selected from H and Me; and
n is an integer selected from 1, 2, 3 and 4.

It may be that the capture composition comprises:
iii) A first liquid phase, said first liquid phase comprising an aqueous solution of an alkali metal salt of a C₂-C₅-aliphatic carboxylic acid; wherein the solution has a concentration such that the molar ratio of salt:water in the range 1:2.5 to 1:5;
iv) A second liquid phase, said second liquid phase comprising a solvent of formula (I) or a mixture of more than one solvent of formula (I):

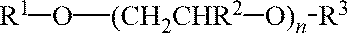

wherein R¹ and R³ are each independently unsubstituted C₁-C₄-alkyl;
R² is independently at each occurrence selected from H and Me; and
n is an integer selected from 1, 2, 3 and 4.

For the absence of doubt, the relative amounts of the components (salt, water, solvent or solvents of formula (I)) is such that the two liquids form separate phases.

Solvents of formula (I) may be partially soluble in water. Thus, the first phase may further comprise the solvent of formula (I) or a mixture of more than one solvent of formula (I). Thus, the first phase may consist of water, the alkali metal salt of a C₂-C₅ carboxylic acid and a solvent of formula (I) or a mixture of solvents of formula (I).

The alkali metal is preferably potassium.

The carboxylic acid is preferably a C₃-C₅ carboxylic acid, e.g. a propionic acid, butyric acid or pentanoic acid. The carboxylic acid may be a C₃-C₄ carboxylic acid. The carboxylic acid may be propionic acid. The carboxylic acid may be a butyric acid, e.g. iso-butyric acid. It may be that the carboxylic acid is not substituted with heteroatom (e.g. O, N, S) containing functional groups.

R² may be at each occurrence H. R² may be at each occurrence methyl.

It may be that R¹ and R³ are each independently unsubstituted C₁-C₃-alkyl. It may be that R¹ and R³ are each independently unsubstituted C₂-C₃-alkyl. It may be that R¹ and R³ are each independently selected from methyl, ethyl and propyl. It may be that R¹ and R³ are each independently selected from ethyl and propyl. R¹ and R³ may be the same. R¹ and R³ may be different. R¹ and R³ may each be ethyl. R¹ and R³ may each be propyl.

n may be an integer selected from 2 and 3. n may be 1. n may be 2. n may be 3. n may be 4.

The second liquid phase may be present in an amount in the range from 10 to 70% of the overall volume of the composition (e.g. the capture composition). The second liquid phase may be present in an amount in the range from 40 to 60% of the overall volume of the composition (i.e. the capture composition). The second liquid phase may be present in an amount in the range from 20 to 50% of the overall volume of the composition (i.e. the capture composition).

The ratio of the first liquid phase to the second liquid phase may be in the range of 9:1 to 1:3 by volume in the overall volume of the composition (e.g. the capture composition). The ratio of the first liquid phase to the second liquid phase may be 3:2 to 2:3 by volume in the overall volume of the composition (e.g. the capture composition). The ratio of the first liquid phase to the second liquid phase may be 4:1 to 1:1 by volume in the overall volume of the composition (e.g. the capture composition).

The gas stream comprising CO₂ may comprise emissions from a combustion process including, but not limited to, energy generation or an industrial process, or as the emissions from a non-combustion industrial process.

Said industrial processes include, but are not limited to, those for which carbon dioxide separation is an integral part of the process such as natural gas sweetening/refining, biogas upgrading, hydrogen production, and syngas production and those for which carbon capture has been proposed as a means of reducing their impact on the ongoing climate crisis such as fermentation, iron and steel production, cement manufacture, glass manufacture, and aluminium smelting.

The gas stream comprising CO₂ may be obtained directly from the atmosphere.

The gas stream comprising CO₂ may be obtained from an enclosed environment including, but not limited to, submarines, and spacecraft.

Typically, contacting the CO₂ with said capture composition may conveniently be achieved by passing the CO₂ containing gas stream through said capture composition, for example with a bubble tray column, or passing the CO₂ containing gas stream alongside said capture composition, for example through a packed column, or using any other process known to those skilled in the art.

The method may further comprise:

releasing said CO₂ by heating the loaded capture composition, and/or by subjecting the loaded capture composition to a stream of stripping gas, for example air, and/or by reducing the pressure above the loaded capture composition to provide a stripped capture composition. This step also provides CO₂.

The method may further comprise:

regenerating the capture composition by cooling and/or increasing the pressure above the stripped capture composition.

Release of the CO₂ typically occurs at temperatures which are in the range of from 0° C. to 300° C., most typically in the range of 40° C. to 200° C., e.g. 60° C. to 150° C.

Release of the CO₂ is conveniently achieved at pressures in the range of from 0 to 150 Bara. In specific embodiments, release of CO₂ may typically be achieved at pressures of around 1 to 5 Bara; in alternative embodiments using pressurised systems, release of CO₂ may typically occur at pressures in the range of from 1 to 30 Bar(abs), most typically around 20 Bar(abs). In some embodiments the release of the CO₂ occurs at a pressure in the range of 0 to 1 Bar(abs).

In certain embodiments of the invention, the first liquid phase is not separated from the second liquid phase prior to the step of releasing the CO₂ from the loaded capture composition. Thus, in certain embodiments, a phase separation step is not performed on the loaded capture composition prior to the step of releasing the CO₂. For the absence of doubt, where CO₂ is released from the loaded capture composition, both the first liquid phase and the second liquid phase are present.

In other embodiments of the invention, a phase separation step may be performed on the loaded capture composition prior to the step of releasing said CO₂. This step comprises separating the first liquid phase from the second liquid phase. The step of releasing said CO₂ is then performed on the first liquid phase only. The second liquid phase may be mixed with fresh or regenerated first liquid phase and recycled to the absorber. Such an embodiment may reduce energy consumption of the CO₂ release step by reducing the mass of liquid that must be heated to CO₂ release temperature.

A particularly useful aspect of the technology is that the release process will generate carbon dioxide in enclosed and/or pressurised systems which will allow for increased pressure and this should reduce further compression requirements for storage applications, with important implications for reductions in overall energy consumption for the complete capture and storage process.

Release of the captured gas from the capture composition via application of heat facilitates regeneration of the capture composition, such that it may be used for further capture and release operations with further CO₂ upon cooling.

Release of the captured CO₂ from the capture composition via the application of a stream of stripping gas, for example air, requires no further regeneration of the solvent before it can be recycled for further capture and release operations. Release of the captured CO₂ via a stream of stripping gas may be further enhanced via the application of heat.

Release of the captured CO₂ from the capture composition via the reduction of pressure in the desorption apparatus requires no further regeneration of the capture composition before it can be recycled except to return the solvent to absorption pressure. Release of the captured CO₂ via a reduction in pressure in the CO₂ release apparatus may be further enhanced via the application of heat.

In a third aspect of the invention is provided a CO₂ capture composition comprising:
i) A first liquid phase, said first liquid phase comprising an aqueous solution of an alkali metal salt of a C₂-C₅ aliphatic carboxylic acid; wherein the solution has a concentration such that the molar ratio of salt:water in the range 1:2.5 to 1:15;
ii) A second liquid phase, said second liquid phase comprising a solvent of formula (I) or a mixture of more than one solvent of formula (I):

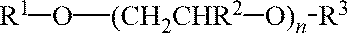

wherein R¹ and R³ are each independently unsubstituted C₁-C₄ alkyl;
R² is independently at each occurrence selected from H and Me; and
n is an integer selected from 1, 2, 3 and 4.

The CO₂ capture composition of the third aspect of the invention may be any capture composition described above in relation to the first aspect of the invention.

In particular, it may be that in the third aspect of the invention, in the first liquid phase comprising an aqueous solution of an alkali metal salt of a C₂-C₅ aliphatic carboxylic acid; the solution has a concentration such that the molar ratio of salt:water in the range 1:2.5 to 1:5.

The invention is further illustrated by the following numbered clauses:
1. A method for capturing CO₂ from a gas stream, the gas stream containing CO₂, the method comprising:
   contacting the gas stream containing CO₂ with a capture composition in a gas-liquid contacting apparatus to generate a loaded capture composition, the capture composition comprising:
      i. a first liquid phase comprising at least one capture reagent; and
      ii. a second liquid phase;
   wherein the second liquid phase is an effective solvent for CO₂ and is chemically inert to CO₂; and
   wherein the motion of the capture composition through the gas-liquid contacting apparatus is induced in such a way that it experiences an acceleration of not more than 5 g.

2. A method of clause 1 wherein the step of contacting the gas stream with the capture composition is carried out in a gas-liquid contacting apparatus selected from: a packed column (with random or structured packings, in co-flow, counter-flow, or cross-flow configurations), a spray tower, a plate or tray column, a stirred tank reactor (in either continuous or batch configuration), a tubular flow reactor (under either laminar or turbulent flow condition), a bubble column reactor, a falling film reactor, or a membrane contactor.

3. A method of clause 1 or clause 2 wherein the first liquid phase comprises an aqueous solution of at least one salt of at least one carboxylic acid.

4. A method of clause 3 wherein the cation of the at least one salt of at least one carboxylic acid is an alkali metal, an alkali earth metal or a mixture thereof.

5. A method of clause 3 or clause 4 wherein the at least one carboxylic acid comprises only carbon, hydrogen and oxygen.

6. A method of any one of clauses 3 to 5 wherein the at least one carboxylic acid is at least one C₁-C₈ aliphatic carboxylic acid.

7. A method of clause 6 wherein the at least one carboxylic acid corresponding to the at least one carboxylate salt are chosen from a list that includes acetic acid, propanoic acid, butyric acid and its branched derivative, pentanoic acid and its branched derivatives, hexanoic acid and its branched derivatives, heptanoic acid and its branched derivatives, and octanoic acid and its branched derivatives.

8. A method of any preceding clause wherein the first liquid phase further comprises at least one carbonate salt.

9. A method of clause 8 wherein the at least one carbonate salt is chosen from a list that includes alkali metal carbonates, alkali earth metal carbonates or a mixture thereof.

10. A method of any preceding clause wherein the first liquid phase further comprises an enzyme.

11. A method of clause 10 wherein the enzyme is a natural carbonic anhydrase or an engineered carbonic anhydrase.

12. A method of any preceding clause wherein the at least one capture reagent is present in the first liquid phase at a concentration in the range 2 M to 15 M.

13. A method of any preceding clause wherein the second liquid phase is an organic solvent.

14. A method of clause 13 wherein the second liquid phase is chosen from a list that includes silicones/siloxanes and ethers.

15. A method of clause 13 wherein the second liquid phase is a solvent selected from: 1,2-dimethoxypropane, 1,2-diethoxypropane, dipropylene glycol dimethyl ether, dipropylene glycol diethyl ether, dipropylene glycol methyl ethyl ether, tripropylene glycol dimethyl ether, tripropylene glycol diethyl ether, tripropylene glycol methyl ethyl ether, 1,2-dimethoxyethane, 1,2-diethoxyethane, ethylene glycol methyl ethyl ether, diethylene glycol dimethyl ether, diethylene glycol diethyl ether, diethylene glycol methyl ethyl ether, triethylene glycol dimethyl ether, triethylene glycol diethyl ether, triethylene glycol methyl ethyl ether, di-isopropyl ether, dibutyl ether, ethyl butyl ether, methyl tert-butyl ether, tetrahydrofuran, 2-methyltetrahydrofuran, octamethyltrisiloxane, decamethyltetrasiloxane, hexamethylcyclotrisiloxane, octamethylcyclotetrasiloxane, butyl acetate, pentyl acetate, pentyl propionate, hexyl propionate, hexyl butyrate, heptyl butyrate, gamma-butyrolactone, gamma-octanoic lactone, 2-pentanone, 3-heptanone, 4-octanone, hexanal, heptanal, octanal, decanal.

16. A method of any preceding clause, wherein the ratio of the first liquid phase to the second liquid phase may be in the range 1:3 to 9:1 by volume.

17. A method of any preceding clause wherein the solubility of CO₂ in the second liquid phase is greater than the solubility of CO₂ in the first liquid phase.

18. A method of any preceding clause, wherein the gas stream comprising CO₂ comprises emissions from a combustion process.

19. A method for reacting a gas from a gas stream with a reactive liquid, the gas stream containing the gas, the method comprising:
contacting the gas stream containing the gas with a composition in a gas-liquid contacting apparatus to generate a product composition, the composition comprising:
   i. a first liquid phase comprising a reagent for reacting with the gas; and
   ii. a second liquid phase;
wherein the second liquid phase is an effective solvent for the gas and is chemically inert to the gas; and
wherein the motion of the composition through the gas-liquid contacting apparatus is induced in such a way that it experiences an acceleration of not more than 5 g.

20. A method of clause 19, wherein the solubility of the gas in the second liquid phase is greater than the solubility of the gas in the first liquid phase.

21. A method of clause 19 or clause 20, wherein the step of contacting the gas stream with the composition is carried out in a gas-liquid contacting apparatus selected from: a packed column (with random or structured packings, in co-flow, counter-flow, or cross-flow configurations), a spray tower, a plate or tray column, a stirred tank reactor (in either continuous or batch configuration), a tubular flow reactor (under either laminar or turbulent flow condition), a bubble column reactor, a falling film reactor, or a membrane contactor.

22. A method for capturing CO₂ from a gas stream, the gas stream containing CO₂, the method comprising:
contacting the gas stream containing CO₂ with a capture composition in a gas-liquid contacting apparatus to generate a loaded capture composition, the capture composition comprising:
   iii. a first liquid phase comprising at least one capture reagent; and
   iv. a second liquid phase capture composition may comprise:
      v) A first liquid phase, said first liquid phase comprising an aqueous solution of an alkali metal salt of a C₂-C₅ aliphatic carboxylic acid; wherein the solution has a concentration such that the molar ratio of salt:water in the range 1:2.5 to 1:5;
      vi) A second liquid phase, said second liquid phase comprising a solvent of formula (I) or a mixture of more than one solvent of formula (I):
         R¹—O—(CH₂CHR²—O)ₙ—R³ (I)
         wherein R¹ and R³ are each independently unsubstituted C₁-C₄ alkyl;
         R² is independently at each occurrence selected from H and Me; and
         n is an integer selected from 1, 2, 3 and 4.

### BRIEF DESCRIPTION OF DRAWINGS

FIG. 1 CO₂ absorption rate as a function of Reynolds number for example 11.

### DETAILED DESCRIPTION OF THE INVENTION

The term “alkyl” refers to a linear or branched hydrocarbon chain. For example, the term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, iso-butyl, sec-butyl, tert-butyl, n-pentyl and n-hexyl. The alkyl group may be unsubstituted.

The term “aliphatic carboxylic acid” refers to a carboxylic acid comprising a CO₂H attached to an alkyl group. It may refer to a carboxylic acid comprising a CO₂H attached to an unsubstituted alkyl group.

For a chemical reaction in a liquid phase involving one or more gaseous reagents, the rate of that reaction can be governed, at least in part, by the availability of the gaseous reagent(s) in said liquid phase. As per Henry’s law, the equilibrium concentration of a gas physically dissolved into a liquid phase is proportional to its partial pressure above the liquid, with the proportionality factor called the Henry’s law constant. In other words, the physical solubility of a gas in a liquid phase is its concentration in the gas phase multiplied by some number, the Henry’s law constant, that accounts for the nature of the gaseous species, the nature of the liquid phase and the temperature at which the measurement is being taken.

For carbon capture applications the concentration of CO₂ in the gas stream to be treated will be dependent on the nature of the process generating said gas stream. For many potential applications of carbon capture technology, the gas stream to be treated is the product of an air-breathing combustion process and as a result may contain relatively little CO₂ (Table 1). Even the waste gases from activities considered to be highly carbon intensive, such as coal-fired power generation (Table 1, Entry 4) or cement manufacture (Table 1, Entry 8), contain relatively low volumetric concentrations of CO₂. Capturing the CO₂ from such sources can be technically challenging, necessitating the use of large scale absorbers, adding to the capital cost of a capture plant, or mechanical process intensification, adding to the energy penalty of the capture process.

TABLE 1

Typical CO₂ content in the flue gas for various potential CCS applications

| Entry | Process | Typical CO₂ Concentration in Stack Gas (%v/v, dry basis) | CO₂ Concentration in Stack Gas (mmol/L, dry basis)^{a} |
|---|---|---|---|
| 1 | Power generation (natural gas fired boiler) | 7-10 | 2.9-4.1 |
| 2 | Power generation (gas turbine) | 3-4 | 1.2-1.6 |
| 3 | Power generation (oil fired boiler) | 11-13 | 4.5-5.3 |
| 4 | Power generation (coal fired boiler) | 12-14 | 4.9-5.7 |
| 5 | Power generation (IGCC -after combustion) | 12-14 | 4.9-5.7 |
| 6 | Oil refinery & petrochemical plant gas fired heaters | 8 | 3.3 |
| 7 | Blast furnace gas | 20-27 | 8.2-11.1 |
| 8 | Cement kiln off-gas | 14-33 | 5.7-13.5 |
| 9 | IGCC (syngas after gasification) | 8-20 | 3.3-8.2 |

^{a}calculated using pV = nRT for p = 1 Bar, T = 293.15 K

An effective solvent for CO₂, as used in this specification, means one which has a Henry’s law constant for CO₂ such that the equilibrium volumetric concentration of CO₂ found in the solvent under a given partial pressure of CO₂ is greater than the volumetric concentration of CO₂ found in the gas phase at the same partial pressure. The critical Henry’s law constant value can be calculated to be approximately 0.041 mol/L/Bar at 20° C. (Table 2). In these circumstances, a solvent with a Henry’s law constant greater than this value may be deemed to be an effective solvent for CO₂, a solvent with a lower Henry’s law constant may not be deemed to be an effective solvent for CO₂.

TABLE 2

Equilibrium concentrations of CO₂ in the liquid phase for solvents with Henry’s law constants at, above and below the critical value to be considered an effective solvent for CO₂ in these circumstances at different partial pressures of CO₂

| Entry | P_{CO2} (kPa) | [CO₂]_{GAS} (mmol/L)^{a} | [CO₂]_{LIQUID} (mmol/L) for Henry’s law constant of | | |
|---|---|---|---|---|---|
| | | | 0.02 mol/L/Bar | 0.041 mol/L/Bar | 0.10 mol/L/Bar |
| 1 | 5 | 2.05 | 1.00 | 2.05 | 5.00 |
| 2 | 15 | 6.15 | 3.00 | 6.15 | 15.00 |
| 3 | 25 | 10.26 | 5.00 | 10.26 | 25.00 |
| 4 | 50 | 20.51 | 10.00 | 20.51 | 50.00 |
| 5 | 100 | 41.03 | 20.00 | 41.02 | 100.00 |

^{a}calculated using pV = nRT for p = 1 Bar, T= 293.15K

The generality of this approach in terms of combinations of different first and second liquid phases will be illustrated with various examples as described below. All of these examples were carried out in our laboratory using our vapour-liquid equilibria (VLE) apparatus which consists of a stirred, jacketed, stainless steel vessel equipped with temperature and pressure sensors. The composition being trialled is brought to test temperature in the vessel and a given partial pressure of CO₂ is added to the gas space above the composition. The CO₂ is supplied from another temperature and pressure monitored reservoir and the partial pressure of CO₂ in the reaction vessel maintained by a regulator. From the temperature and pressure data of the CO₂ reservoir recorded as a function of time, the rate of CO₂ absorption and overall amount of CO₂ absorbed can be calculated. For each of the examples below, the salt(s) and water were combined in the desired ratio and the organic solvent added until a second liquid phase emerged. The composition was then run in the VLE apparatus twice; firstly with a single phase composition in which the organic solvent is present at an amount in which it is completely dissolved in the aqueous salt solution then secondly with the organic solvent present at an amount at which it forms a second liquid phase as well as being dissolved in the aqueous salt solution. In all cases, the runs in which the composition has two liquid phases were carried out with a 1:1 liquid phase ratio by volume. Absorption rates are reported as moles of CO₂ absorbed per litre of composition per Bar of CO₂ partial pressure per hour.

Example 1: Potassium propionate and water in a molar ratio of 1 to 3 with methyl isobutyrate. Single phase absorption experiment exhibited a maximum absorption rate of 2 mol/L/Bar/h. Two liquid phase absorption experiment exhibited a maximum absorption rate of 5 mol/L/Bar/h.

Example 2: Potassium acetate and water in a molar ratio of 1 to 3 with diethylene glycol dimethyl ether. Single phase absorption experiment exhibited a maximum absorption rate of 2.5 mol/L/Bar/h. Two liquid phase absorption experiment exhibited a maximum absorption rate of 6.5 mol/L/Bar/h.

Example 3: 7 M Potassium acetate in water with methyl isobutyrate. Single phase absorption experiment exhibited a maximum absorption rate of 2 mol/L/Bar/h. Two liquid phase absorption experiment exhibited a maximum absorption rate of 6.5 mol/L/Bar/h.

Example 4: Potassium acetate and water in a molar ratio of 1 to 3 with polymethylhydrosiloxane. Single phase absorption experiment exhibited a maximum absorption rate of 1.5 mol/L/Bar/h. Two liquid phase absorption experiment exhibited a maximum absorption rate of 4.5 mol/L/Bar/h.

Example 5: Potassium propionate and water in a molar ratio of 1 to 3.25 with 2-pentanone. Single phase absorption experiment exhibited a maximum absorption rate of 4 mol/L/Bar/h. Two liquid phase absorption experiment exhibited a maximum absorption rate of 8 mol/L/Bar/h.

Example 6: Potassium propionate and water in a molar ratio of 1 to 3.25 with butyl acetate. Single phase absorption experiment exhibited a maximum absorption rate of 4 mol/L/Bar/h. Two liquid phase absorption experiment exhibited a maximum absorption rate of 8 mol/L/Bar/h.

Example 7: Potassium propionate and water in a molar ration of 1 to 3.25 with cyclohexanone. Single phase absorption experiment exhibited a maximum absorption rate of 2.5 mol/L/Bar/h. Two liquid phase absorption experiment exhibited a maximum absorption rate of 5.5 mol/L/Bar/h.

Example 8: Potassium propionate and water in a molar ratio of 1 to 3.25 with diethylene glycol diethyl ether. Single phase absorption experiment exhibited a maximum absorption rate of 0.5 mol/L/Bar/h. Two liquid phase absorption experiment exhibited a maximum absorption rate of 7 mol/L/Bar/h.

Example 9: Potassium propionate and water in a molar ration of 1 to 3.25 with ethyl acetate. Single phase absorption experiment exhibited a maximum absorption rate of 4 mol/L/Bar/h. Two liquid phase absorption experiment exhibited a maximum absorption rate of 6.5 mol/L/Bar/h.

Example 10: 0.6 M Potassium hexanoate and 4 M potassium carbonate in water with cyclohexanone. Single phase absorption experiment exhibited a maximum absorption rate of 3.5 mol/L/Bar/h. Two liquid phase absorption experiment exhibited a maximum absorption rate of 6 mol/L/Bar/h.

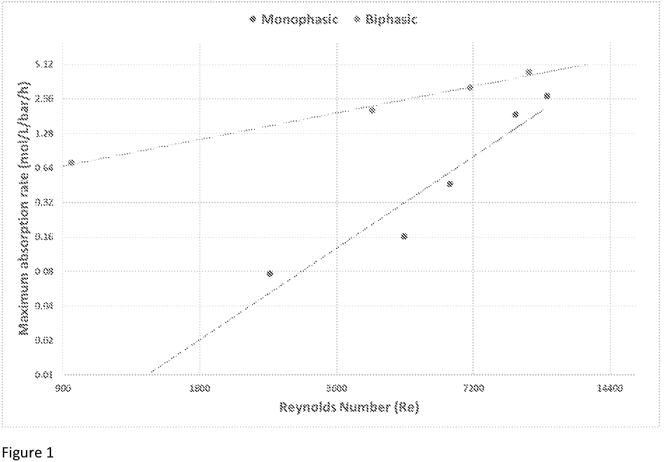

Example 11: This final example provided herein is to demonstrate that the effect is general across a wide range of stirring rates, i.e. mechanical inputs. The experiments were run in the same way as in examples 1 through 10 except that for this final example, the absorption was run multiple times with the stirring rate being varied between runs. The Reynolds number was calculated for each run and a graph plotted (FIG. 1) showing the maximum absorption rate (in mol/L/Bar/h as above) as a function of Reynolds number. As can be seen, the accelerating effect of the second liquid phase is general across a wide range of stirring rates/Reynolds number. It should be noted that the axes of the graph are logarithmic so that even at the highest Reynolds number achievable in our apparatus (Re = ca. 10,000), the rate of absorption into the biphasic system was approximately twice the rate of absorption into the monophasic system.

The composition used for this final example was potassium propionate and water in a molar ratio of 1 to 3.25 with butyl acetate as the second liquid phase.

1. A method for capturing CO₂ from a gas stream, the gas stream containing CO₂, the method comprising:
contacting the gas stream containing CO₂ with a capture composition in a gas-liquid contacting apparatus to generate a loaded capture composition, the capture composition comprising:
i. a first liquid phase comprising at least one capture reagent; and
ii. a second liquid phase;
wherein the second liquid phase is an effective solvent for CO₂ and is chemically inert to CO₂;
wherein the first liquid phase comprises an aqueous solution of at least one salt of at least one carboxylic acid; and
wherein the method further comprises releasing said CO₂ from the loaded capture composition.

2. A method of claim 1, wherein the step of contacting the gas stream with the capture composition is carried out in a gas-liquid contacting apparatus selected from: a packed column (with random or structured packings, in co-flow, counter-flow, or crossflow configurations), a spray tower, a plate or tray column, a stirred tank reactor (in either continuous or batch configuration), a tubular flow reactor (under either laminar or turbulent flow condition), a bubble column reactor, a falling film reactor, or a membrane contactor.

3. A method of claim 1, wherein the cation of the at least one salt of at least one carboxylic acid is an alkali metal, an alkali earth metal or a mixture thereof.

4. A method of claim 3, wherein the at least one carboxylic acid comprises only carbon, hydrogen and oxygen.

5. A method of claim 4, wherein the at least one carboxylic acid is at least one C₁-C₈ aliphatic carboxylic acid.

6. A method of claim 5, wherein the at least one carboxylic acid corresponding to the at least one carboxylate salt are chosen from a list that includes acetic acid, propanoic acid, butyric acid and its branched derivative, pentanoic acid and its branched derivatives, hexanoic acid and its branched derivatives, heptanoic acid and its branched derivatives, and octanoic acid and its branched derivatives.

7. A method of claim 1, wherein the first liquid phase further comprises at least one carbonate salt.

8. A method of claim 7, wherein the at least one carbonate salt is chosen from a list that includes alkali metal carbonates, alkali earth metal carbonates or a mixture thereof.

9. A method of claim 1, wherein the first liquid phase further comprises an enzyme.

10. A method of claim 9, wherein the enzyme is a natural carbonic anhydrase or an engineered carbonic anhydrase.

11. A method of claim 1, wherein the at least one capture reagent is present in the first liquid phase at a concentration in the range 2 M to 15 M.

12. A method of claim 1, wherein the second liquid phase is an organic solvent.

13. A method of claim 12, wherein the second liquid phase is chosen from a list that includes silicones/siloxanes and ethers.

14. A method of claim 12, wherein the second liquid phase is a solvent selected from: 1,2-dimethoxypropane, 1,2-diethoxypropane, dipropylene glycol dimethyl ether, dipropylene glycol diethyl ether, dipropylene glycol methyl ethyl ether, tripropylene glycol dimethyl ether, tripropylene glycol diethyl ether, tripropylene glycol methyl ethyl ether, 1,2-dimethoxyethane, 1,2-diethoxyethane, ethylene glycol methyl ethyl ether, diethylene glycol dimethyl ether, diethylene glycol diethyl ether, diethylene glycol methyl ethyl ether, triethylene glycol dimethyl ether, triethylene glycol diethyl ether, triethylene glycol methyl ethyl ether, di-isopropyl ether, dibutyl ether, ethyl butyl ether, methyl tert-butyl ether, tetrahydrofuran, 2-methyltetrahydrofuran, octamethyltrisiloxane, decamethyltetrasiloxane, hexamethylcyclotrisiloxane, octamethylcyclotetrasiloxane, butyl acetate, pentyl acetate, pentyl propionate, hexyl propionate, hexyl butyrate, heptyl butyrate, gamma-butyrolactone, gamma-octanoic lactone, 2-pentanone, 3-heptanone, 4-octanone, hexanal, heptanal, octanal, decanal.

15. A method of claim 1, wherein the ratio of the first liquid phase to the second liquid phase may be in the range 1:3 to 9:1 by volume.

16. A method of claim 1, wherein the physical solubility of CO₂ in the second liquid phase is greater than the physical solubility of CO₂ in the first liquid phase.

17. A method of claim 1, wherein the gas stream comprising CO₂ comprises emissions from a combustion process.

18. A method of claim 1, wherein said CO₂ is released by:
(i) heating the loaded capture composition; and/or
(ii) subjecting the loaded capture composition to a stream of stripping gas, for example air; and/or
(iii)reducing the pressure above the loaded capture composition to provide a stripped capture composition.

19. A method for capturing CO₂ from a gas stream, the gas stream containing CO₂, the method comprising:
contacting the gas stream containing CO₂ with a capture composition in a gas-liquid contacting apparatus to generate a loaded capture composition, the capture composition comprising:
i. a first liquid phase, said first liquid phase comprising an aqueous solution of an alkali metal salt of a C₂-C₅ aliphatic carboxylic acid; wherein the solution has a concentration such that the molar ratio of salt:water in the range 1:2.5 to 1:15; and
ii. a second liquid phase, said second liquid phase comprising a solvent of formula (I) or a mixture of more than one solvent of formula (I): 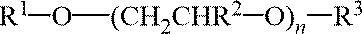
wherein R¹ and R³ are each independently unsubstituted C₁-C₄ alkyl;
R² is independently at each occurrence selected from H and Me; and
n is an integer selected from 1, 2, 3 and 4.

20. A method of claim 19, wherein the molar ratio of salt:water is in the range of 1:2.5 to 1:5.
